(19) United States
(12) Patent Application Publication
(10) Pub. No.: US 2014/0066374 A1
(43) Pub. Date: Mar. 6, 2014

(54) Simultaneous Delivery of Receptors and/or Co-Receptors for Growth Factor Stability and Activity

(71) Applicants: Elazer R. Edelman, Brookline, MA (US); Aaron B. Baker, Cambridge, MA (US)

(72) Inventors: Elazer R. Edelman, Brookline, MA (US); Aaron B. Baker, Cambridge, MA (US)

(73) Assignee: MASSACHUSETTS INSTITUTE OF TECHNOLOGY, Cambridge, MA (US)

(21) Appl. No.: 13/887,722

(22) Filed: May 6, 2013

Related U.S. Application Data
(63) Continuation of application No. 12/389,765, filed on Feb. 20, 2009, now abandoned.
(60) Provisional application No. 61/030,419, filed on Feb. 21, 2008.

(51) Int. Cl. A61K 38/18; A61K 38/17

(52) U.S. Cl. CPC A61K 38/1825; A61K 38/179; A61K 38/177
USPC 514/9.1; 435/375; 435/377

(57) ABSTRACT

The compositions and methods of the present invention relate to the co-delivery of a molecule and a polypeptide to cells to improve the therapeutic efficacy of the molecules. In one embodiment of the invention, the invention may improve delivery of growth factors by co-delivering these growth factors with their receptors and co-receptors, such as syndecans. Co-delivery of growth factors with syndecans, for example, may protect growth factors from proteolysis, enhance their activity, and target the growth factors to the cell surface to facilitate growth factor signaling. This novel approach to growth factor therapy could be extended to other systems and growth factors enabling the enhancement of multiple signaling pathways to achieve a desired therapeutic outcome.

### CROSS REFERENCE TO RELATED APPLICATIONS

This application claims priority to and the benefit under 35 U.S.C. Section 119(e) of U.S. Provisional Patent Application Ser. No. 61/030,419, filed Feb. 21, 2008, the entire disclosure of which is incorporated by reference herein.

### FIELD OF THE INVENTION

The present invention concerns improving the delivery of therapeutic agents to cells. Specifically, the invention relates to the co-delivery of ligands, such as growth factors, with their receptors or co-receptors in order to protect the ligands from proteolysis and to improve proper localization of the ligand.

### BACKGROUND OF THE INVENTION

Chronic myocardial and peripheral ischemic disease affect about 27 million patients in the United States and are one of the leading causes of morbidity and mortality in developed countries. Current therapy for ischemia consists of drug-based interventions to slow progression of vascular disease, endovascular stent placement and surgical bypass of stenosed arteries. While these treatments can delay or temporarily reduce ischemia, none address the fundamental issue of compromised perfusion due to dysfunctional microvasculature. In the continuing search for new therapies for both myocardial and peripheral ischemia, the therapeutic delivery of growth factors has received much attention both in basic and clinical studies. While this modality for achieving therapeutic angiogenesis has shown promising results in early studies, the implementation of this strategy in humans has met with only mixed or negative results. One potential reason for this lack of clinical efficacy is the loss of growth factor surface receptors and co-receptors as result of the existing disease state. Fibroblast growth factor-2 (FGF-2) was one of the first growth factors to be tested for clinical efficacy for myocardial and peripheral ischemia. The binding of this growth factor to its receptor is weak and reversible in the absence of stabilization by heparan sulfate proteoglycans (HSPGs). Disease states are known to modulate HSPGs by regulating both the amount and structure of these complex molecules. If HSPG chains are reduced or dysfunctional in vascular disease then no amount of growth factor delivered will achieve effective signaling and stimulation of revascularization. Here we present a novel solution to this problem by using liposome-embedded syndecan-4 to enhance FGF-2 activity in-vitro and in an animal model of peripheral ischemia.

While the induction of angiogenesis is an appealing concept for the treatment of ischemic disease, the implementation of this therapeutic strategy has proved troublesome. Clinical trials for treating ischemia with growth factors or by delivering growth factor genes have met with mixed results. In these trials, relatively large amounts of growth factors were applied to tissue with the expectation of achieving a high degree of revascularization. Cells are well known to have negative feedback mechanisms, including receptor and co-receptor down regulation that can lead to insensitivity to growth factor stimulation. Furthermore, in the clinical setting growth factor therapy is always administered in the presence of an underlying disease state. In the case of ischemia, these often include a combination of atherosclerosis, diabetes and hypertension. These disease states can also alter receptor/co-receptor dynamics and lead to cell insensitivity to growth factor therapy. By definition the presence of ischemia itself implies a defeat of the natural revascularization processes mediated in part by overproduction of endogenous growth factors. To address this problem we developed a novel drug delivery strategy to maintain high levels of co-receptor associated with the cell surface. Our formulation consisted of recombinant syndecan-4, a cell surface HSPG and co-receptor for FGF-2, embedded in liposomes and delivered concomitantly with FGF-2 (illustrated in FIG. 1a). Syndecan-4 is a cell surface heparan sulfate proteoglycan (HSPG) that can stabilize the interaction between FGF-2 and the FGFR-1 receptor in endothelial cells. We hypothesized that co-delivery of a lipid embedded co-receptor would enhance the effectiveness of FGF-2, increasing the cells ability to respond to FGF-2 in addition to providing a stimulatory ligand. We show here that this drug delivery strategy enhances FGF-2 mediated proliferation, migration and tube formation in-vitro. In addition, this formulation increased revascularization in a hind limb ischemia model in rats in comparison to FGF-2 alone. This novel approach to growth factor therapy could be extended to other systems and growth factors enabling the enhancement of multiple signaling pathways to achieve a desired therapeutic outcome.

A large body of scientific work has been performed to evaluate potential in the pursuit of efficacious revascularization therapy. These include delivery of the growth factor protein, viral delivery of growth factor/transcription factor genes, induction/mobilization of endogenous endothelial progenitor cells and implantation of bone marrow or progenitor cells. Each of these strategies has shown some promise in early phase experimental work and animal models but to date none have shown efficacy in large, randomized clinical trials. In particular, the FIRST trial, a phase II, randomized and double blinded trial, using FGF-2 as sole therapy showed no improvement in myocardial perfusion or exercise treadmill testing (ETT) despite promising early studies. Similarly, a phase II/III trial (the VIVA trial) found no improvement in comparison to placebo. Clinical trials of adenoviral delivered DNA have shown no improvement in ETT but some increases in myocardial perfusion. Cell therapy is a relatively new and controversial strategy based on delivering endothelial or stem cells. Trials of this type of therapy have shown promise but no large clinical trials evaluating this strategy have been performed. An inherent assumption of all of these therapeutic strategies is that ischemic tissue is capable of mounting an appropriate neovascularization response to an angiogenic/arteriogenic stimulus. The work presented here aimed to facilitate angiogenesis even with a compromised receptor and co-receptor system. While this represents only one potential category of a defective angiogenic system, the enhancement of angiogenesis found in our work would suggest that there is a benefit to overcoming this aspect of inherent cellular and pathophysiologically induced growth factor desensitization.

Prior to this work people have only used growth factors or transfected growth factor receptors into cells or tissues to try to enhance neovascularization. The present invention is directed to improving delivery of growth factors by co-delivering these growth factors with their receptors and co-receptors directly to the cell membrane by means of a flexible carrier such as a liposome. Co-delivery of growth factors with receptors or co-receptors may protect growth factors from proteolysis, enhance their activity, and target the growth factors to the cell surface to facilitate growth factor signaling. In one embodiment of the invention, the syndecans may function as co-receptors in this new paradigm of drug delivery as they bind many growth factors through their heparan sulfate chains and are known to be active participants in the signaling pathways of growth factors associated with angiogenesis (e.g. Fibroblast Growth Factor (FGF) and Vascular Endothelial Cell Growth Factor (VEGF)).

### SUMMARY OF THE INVENTION

In one aspect, the present invention relates to a method for modulating the therapeutic efficacy of a molecule. The method comprises providing a flexible carrier with at least one polypeptide that comprises a transmembrane region embedded therein. The method further comprises co-delivering to a cell (i) a molecule capable of selectively binding the at least one polypeptide and (ii) the flexible carrier into which the at least one polypeptide is embedded. Co-delivery results in modulation of the therapeutic efficacy of said molecule.

In another aspect, the invention relates to a method for modulating cell signaling, cell secretion, cell proliferation, cell migration and/or cell differentiation. The method comprises providing a flexible carrier with at least one polypeptide embedded therein, said at least one polypeptide comprising a transmembrane region, and co-delivering to a cell (i) a molecule capable of selectively binding the at least one polypeptide and (ii) the flexible carrier into which the at least one polypeptide is embedded. According to the method, the co-delivery results in modulation of cell signaling, cell proliferation, cell migration and/or cell differentiation.

In one embodiment, the modulated cell signaling, cell proliferation, cell migration and/or cell differentiation results in modulation, control or regulation of cell, organ, or tissue preservation, repair, replacement, or regeneration, including processes that involve hypoxia, angiogenesis, wound healing, ischemia, apoptosis, or inflammation, including those of acute, reactive, autoimmune and chronic nature wound, cell, organ and tissue repair, wherein applicable systems include but are not isolated to repair of cosmetic or surgical wounds from superficial skin incisions, deep tissue excision or biopsies of cells, tissue or organs of the skin, hair, bones and joints (including the arthritites, degenerative, metabolic and infectious diseases), brain, eye (that might also include corneal graft rejection, neovascular glaucoma, retrolental fibroplasia, epidemic keratoconjunctivitis), ear, nose, tracheobronchial tree, oropharynx, teeth, gastrointestinal tract, salivary glands, liver, spleen, pancreas, gall bladder, genitourinary tract, kidney, bladder, uterus, ovaries, prostate accidental or unintended injury, fracture, laceration or noxious exposure diseases of the neural systems that involve tissue preservation, repair, replacement or regeneration including amyotrophic lateral sclerosis, Alzheimer's disease, Parkinson's disease Huntington's disease, ischemic stroke, acute brain injury, acute spinal chord injury, multiple sclerosis and peripheral nerve injury regeneration and guidance vascular repair and control of aneurysms, hemangiomas, thrombosis, spasm, intimal hyperplasia and restenosis, myocardial hypertrophy and remodeling, weight loss/fat metabolism, congenital dysplasia, malformation, altered development of cells, tissues and/or organs and their preservation, repair, replacement or regeneration acquired infectious diseases including bacterial, viral, parasitic and protozoal origin, and of AIDS/HIV, hematologic, neoplastic, metastatic and dysplastic diseases including cancer of solid organs, circulating blood, bone marrow and blood precursor cells and when used alone or in concert with other device, pharmacolologic, cell-based or tissue engineered therapies, including combination products and stem cell based therapies.

In another embodiment, the co-delivery of the molecule and of the flexible carrier into which the at least one polypeptide is embedded occurs simultaneously. In other embodiments, the at least one polypeptide comprises a syndecan or fragment thereof, a wild-type or mutant syndecan-1 or a fragment thereof, a wild-type or mutant syndecan-2 or a fragment thereof, a wild-type or mutant syndecan-3 or a fragment thereof, a wild-type or mutant syndecan-4 or a fragment thereof. In other embodiments, the mutant syndecan comprises a mutation in a glycosaminoglycan-attachment site and/or a mutation in a residue recognized or cleaved by a sheddase, wherein the mutation decreases the ability of said mutant syndecan to be cleaved as compared to a corresponding wild-type syndecan. In yet another embodiment, the method comprises an additional step of providing a heparanase to the extracellular surface of the cell.

In other embodiments, the at least one polypeptide is a growth factor receptor, such as an immunomodulatory growth factor receptor, a neuropilin, a thrombospondin receptor such as CD36. In other embodiments, the molecule is a growth factor, a cytokine, and/or a thrombospondin. In another embodiment of the method of the invention, the flexible carrier comprises two polypeptides, wherein the two polypeptides are a growth factor receptor and a syndecan, and further wherein the molecule is a growth factor. In another embodiment, the flexible carrier comprises lipids and proteins. In another embodiment, the ratio of lipids to proteins is in the range from 20:80 to 80:20. In yet another embodiment, the flexible carrier comprising lipids and proteins is a liposome.

In another aspect, the invention relates to a method for modulating cell signaling, cell secretion, cell proliferation, cell migration and/or cell differentiation, the method comprising providing a liposome comprising syndecan-4 and co-delivering to a cell (i) fibroblast growth factor (FGF) and (ii) the liposome comprising syndecan-4, wherein the co-delivery results in modulated signaling, secretion, proliferation, migration and/or differentiation of the cell. In one embodiment, the modulated cell signaling, cell proliferation, cell migration and/or cell differentiation results in modulation, control or regulation of cell, organ, or tissue preservation, repair, replacement, or regeneration, including processes that involve hypoxia, angiogenesis, wound healing, ischemia, apoptosis, or inflammation, including those of acute, reactive, autoimmune and chronic nature wound, cell, organ and tissue repair, wherein applicable systems include but are not isolated to repair of cosmetic or surgical wounds from superficial skin incisions, deep tissue excision or biopsies of cells, tissue or organs of the skin, hair, bones and joints (including the arthritites, degenerative, metabolic and infectious diseases), brain, eye (that might also include corneal graft rejection, neovascular glaucoma, retrolental fibroplasia, epidemic keratoconjunctivitis), ear, nose, tracheobronchial tree, oropharynx, teeth, gastrointestinal tract, salivary glands, liver, spleen, pancreas, gall bladder, genitourinary tract, kidney, bladder, uterus, ovaries, prostate accidental or unintended injury, fracture, laceration or noxious exposure diseases of the neural systems that involve tissue preservation, repair, replacement or regeneration including amyotrophic lateral sclerosis, Alzheimer's disease, Parkinson's disease Huntington's disease, ischemic stroke, acute brain injury, acute spinal chord injury, multiple sclerosis and peripheral nerve injury regeneration and guidance vascular repair and control of aneurysms, hemangiomas, thrombosis, spasm, intimal hyperplasia and restenosis, myocardial hypertrophy and remodeling, weight loss/fat metabolism, congenital dysplasia, malformation, altered development of cells, tissues and/or organs and their preservation, repair, replacement or regeneration acquired infectious diseases including bacterial, viral, parasitic and protozoal origin, and of AIDS/HIV, hematologic, neoplastic, metastatic and dysplastic diseases including cancer of solid organs, circulating blood, bone marrow and blood precursor cells and when used alone or in concert with other device, pharmacolologic, cell-based or tissue engineered therapies, including combination products and stem cell based therapies.

In another aspect, the invention relates to a method for enhancing wound healing, comprising providing to a subject a flexible carrier with a syndecan and/or a growth factor receptor embedded therein, and co-delivering to the subject (i) a growth factor capable of selectively binding the syndecan and/or the growth factor receptor and (ii) the flexible carrier into which the syndecan and/or the growth factor receptor is/are embedded, wherein the co-delivery results in enhancement of wound healing. In one embodiment, the wound is a diabetic foot ulcer (DFU).

In yet another aspect, the invention relates to a method for enhancing angiogenesis, comprising providing to a subject a flexible carrier with a syndecan and/or a growth factor receptor embedded therein, and co-delivering to said subject (i) a growth factor capable of selectively binding the syndecan and/or the growth factor receptor and (ii) the flexible carrier into which the syndecan and/or the growth factor receptor is/are embedded, wherein the co-delivery results in enhancement of angiogenesis. In one embodiment, the subject has peripheral or myocardial ischemia.

In one aspect, the invention relates to a method for producing a recombinant syndecan polypeptide with improved growth factor signaling enhancement properties comprising (a) transfecting a cancer cell line with a polynucleotide comprising a syndecan gene, and (b) purifying a syndecan polypeptide from the cancer cell line, wherein the syndecan polypeptide has improved growth factor signaling enhancement properties. In one embodiment, the method further comprises the step of providing a cell with the purified recombinant syndecan polypeptide.

In another aspect, the present invention provides for a composition comprising a flexible carrier comprising at least one polypeptide embedded therein, wherein the polypeptide is selected from the group consisting of syndecan-1, syndecan-2, syndecan-3, syndecan-4, and a growth factor receptor and further comprises a growth factor is selectively bound to the polypeptide, wherein the composition is capable of modulating cell proliferation, cell secretion, cell migration and/or cell differentiation. In another embodiment, the modulated cell signaling, cell proliferation, cell migration and/or cell differentiation results in modulation, control or regulation of cell, organ, or tissue preservation, repair, replacement, or regeneration, including processes that involve hypoxia, angiogenesis, wound healing, ischemia, apoptosis, or inflammation, including those of acute, reactive, autoimmune and chronic nature wound, cell, organ and tissue repair, wherein applicable systems include but are not isolated to repair of cosmetic or surgical wounds from superficial skin incisions, deep tissue excision or biopsies of cells, tissue or organs of the skin, hair, bones and joints (including the arthritites, degenerative, metabolic and infectious diseases), brain, eye (that might also include corneal graft rejection, neovascular glaucoma, retrolental fibroplasia, epidemic keratoconjunctivitis), ear, nose, tracheobronchial tree, oropharynx, teeth, gastrointestinal tract, salivary glands, liver, spleen, pancreas, gall bladder, genitourinary tract, kidney, bladder, uterus, ovaries, prostate accidental or unintended injury, fracture, laceration or noxious exposure diseases of the neural systems that involve tissue preservation, repair, replacement or regeneration including amyotrophic lateral sclerosis, Alzheimer's disease, Parkinson's disease Huntington's disease, ischemic stroke, acute brain injury, acute spinal chord injury, multiple sclerosis and peripheral nerve injury regeneration and guidance vascular repair and control of aneurysms, hemangiomas, thrombosis, spasm, intimal hyperplasia and restenosis, myocardial hypertrophy and remodeling, weight loss/fat metabolism, congenital dysplasia, malformation, altered development of cells, tissues and/or organs and their preservation, repair, replacement or regeneration acquired infectious diseases including bacterial, viral, parasitic and protozoal origin, and of AIDS/HIV, hematologic, neoplastic, metastatic and dysplastic diseases including cancer of solid organs, circulating blood, bone marrow and blood precursor cells and when used alone or in concert with other device, pharmacolologic, cell-based or tissue engineered therapies, including combination products and stem cell based therapies. In another embodiment, the flexible carrier is a liposome.

In another aspect, the present invention relates to a composition comprising a liposome into which syndecan-4 is embedded, wherein FGF is selectively bound to syndecan-4, and wherein the composition is capable of modulating cell signaling, cell secretion, cell proliferation, cell migration and/or cell differentiation. In one embodiment, the modulated cell signaling, cell proliferation, cell migration and/or cell differentiation results in modulation, control or regulation of cell, organ, or tissue preservation, repair, replacement, or regeneration, including processes that involve hypoxia, angiogenesis, wound healing, ischemia, apoptosis, or inflammation, including those of acute, reactive, autoimmune and chronic nature wound, cell, organ and tissue repair, wherein applicable systems include but are not isolated to repair of cosmetic or surgical wounds from superficial skin incisions, deep tissue excision or biopsies of cells, tissue or organs of the skin, hair, bones and joints (including the arthritites, degenerative, metabolic and infectious diseases), brain, eye (that might also include corneal graft rejection, neovascular glaucoma, retrolental fibroplasia, epidemic keratoconjunctivitis), ear, nose, tracheobronchial tree, oropharynx, teeth, gastrointestinal tract, salivary glands, liver, spleen, pancreas, gall bladder, genitourinary tract, kidney, bladder, uterus, ovaries, prostate accidental or unintended injury, fracture, laceration or noxious exposure diseases of the neural systems that involve tissue preservation, repair, replacement or regeneration including amyotrophic lateral sclerosis, Alzheimer's disease, Parkinson's disease Huntington's disease, ischemic stroke, acute brain injury, acute spinal chord injury, multiple sclerosis and peripheral nerve injury regeneration and guidance vascular repair and control of aneurysms, hemangiomas, thrombosis, spasm, intimal hyperplasia and restenosis, myocardial hypertrophy and remodeling, weight loss/fat metabolism, congenital dysplasia, malformation, altered development of cells, tissues and/or organs and their preservation, repair, replacement or regeneration acquired infectious diseases including bacterial, viral, parasitic and protozoal origin, and of AIDS/HIV, hematologic, neoplastic, metastatic and dysplastic diseases including cancer of solid organs, circulating blood, bone marrow and blood precursor cells and when used alone or in concert with other device, pharmacolologic, cell-based or tissue engineered therapies, including combination products and stem cell based therapies.

In another aspect, the present invention provides for a mutant syndecan comprising a mutation in a glycosaminoglycan-attachment site, wherein the shed mutant syndecan modulates cell signaling, cell secretion, cell proliferation, cell migration and/or cell differentiation. In one embodiment, the mutant syndecan contains no glycosaminoglycan-attachment sites.

In another aspect, the present invention provides for a mutant syndecan comprising a mutation in a residue recognized and/or cleaved by a sheddase, wherein themutation decreases the ability of the mutant syndecan to be cleaved as compared to a corresponding wild-type syndecan.

### BRIEF DESCRIPTION OF THE FIGURES

FIG. 1: Concept and analysis of syndecan-4 embedded liposome formulation. (a) Diagram of syndecan-4 liposome constructs. Purified syndecan-4 was embedded in liposomes and co-delivered with FGF-2 to enhance growth factor activity. (b) Transmission electron micrographs of liposome embedded syndecan-4. Bar=500 nm. (c) Syndecan-4 protein concentration alters final liposome diameter. Lines from left to right: P20:L80; P40:L80; P60:L40; P80:L20. (d) Liposome-embedded syndecan-4 causes increased ¹²⁵I-labeled FGF-2 uptake kinetics. Columns from left to right at each time point: FGF-2 alone; FGF-2 with liposomes; FGF-2 with syndecan-4; FGF-2 with syndecan-4 proteoliposomes. (e) Liposome embedding prolongs ³H-labeled syndecan-4 presence in the media after 30 min (p=0.02) or 60 min (p=1×10₋₄). Syndecan-4 alone is shown with black bars and syndecan-4 proteoliposomes are shown with white bars. Statistically significant difference between samples (p<0.05).

FIG. 2. Liposome embedded syndecan-4 enhanced FGF-2 stimulation of endothelial proliferation and migration. (a) Dose response curve for various concentration of liposome/syndecan-4 formulation. Black bars are samples without FGF-2 and white bars are samples with 10 ng/ml FGF-2 added. (b) Optimization of lipid to protein ratio to enhance FGF-2 induced proliferation. (c) Wound healing assay showing wound edge migration under various treatments. Bar=100 μm. (d) Quantitative analysis of wound edge closure following wounding and in the presence of various treatments.

FIG. 3. Liposome/syndecan-4 enhances in-vitro tube formation in combination with FGF-2. (a) Phase contrast micrographs of endothelial cells in Matrigel. Bar=200 μm. (b) Tube length of endothelial tubes formed after 12 hours. (c) Number of branch points in endothelial networks after 12 hours. (d) Length of tubes in the endothelial network following 24 hours of incubation. (e) Branch points in endothelial networks after 24 hours of treatment.

FIG. 4. Histological examination of the ischemic hind limb muscle after femoral artery ligation and concomitant treatment with various drug formulations. (a) Routine histological staining revealed reduced ischemic changes following treatment with FGF-2 in combination with syndecan-4 alone or with liposome embedded syndecan-4. Hematoxylin and eosin (H&E) staining has a size bar=100 μm. PECAM-1 size bar=50 μm. (b) Quantification of large vessels number per field of view. Statistically different from FGF group (p<0.05). Statistically different from all other groups (p<0.05). (c) Quantification of capillaries number per field of view. Statistically different from FGF group (p<0.05).

### DETAILED DESCRIPTION OF THE INVENTION

The compositions and methods of the present invention relate to the co-delivery of a molecule and a polypeptide to cells to modulate the therapeutic efficacy of the molecules. The therapeutic efficacy of a molecule may be any effect the molecule has on a cell. Modulating the therapeutic efficacy of a molecule may include, but is not limited to, increasing therapeutic efficacy, decreasing therapeutic efficacy, increasing the number/and or type of cells affected by the molecule, or changing the effect the molecule has on the cell, for example.

As used herein, a molecule may be any object capable of selectively binding to the polypeptide(s) of the invention. Selective binding refers to an interaction between two molecules which can be assayed in a number of ways known to those skilled in the art, for example, but not limited to yeast-two-hybrid assays or co-immunoprecipitation experiments. According to the invention, a molecule may be a drug, compound, nucleotide, or polypeptide, for example. A polypeptide may be any chain comprised of more than one amino acid. The term polypeptide may be used interchangeably with protein.

A flexible carrier may be any material suitable for delivering a transmembrane polypeptide to the membrane of a cell. Modifications may be made to a flexible carrier to increase the efficiency with which the flexible carrier delivers a polypeptide to a cell, for example, by changing the ratio of materials present in the flexible carrier. A flexible carrier may be a lipid-based vehicle. For example, a flexible carrier may comprise lipids suitable for delivering one or more polypeptides to a cell, preferably by means of the fusion of the flexible carrier with the cell. A lipid-based vehicle may comprise phospholipids, glycolipids or steroids, for example. A lipid-based vehicle that comprises phospholipids may exist as a monolayer or a bilayer. Modifications may be made to a lipid-based vehicle to increase the efficiency with which the lipid-based vehicle fuses with a cell, for example, by changing the lipid content. A lipid-based vehicle may be a micelle or a bacterial or red cell ghost. A lipid-based vehicle may be vesicles or membrane fragments of transgenic cells. In a preferred embodiment, a flexible carrier may be a liposome. A liposome is a general category of vesicle which may comprise one or more lipid bilayers surrounding an aqueous space. Liposomes include unilamellar vesicles composed of a single membrane or a lipid bilayer, and multilamellar vesicles (MLVs) composed of many concentric membranes (or lipid bilayers). Methods for liposome production are well known in the art (see U.S. Pat. No. 6,248,353, for example).

A flexible carrier may also have few or no lipid components. Examples of non-lipid transmembrane polypeptide carriers are described in U.S. Pat. No. 6,492,501, the contents of which are hereby incorporated by reference. A flexible carrier may comprise amphiphilic peptide polymers such as peptitergents, or modified amphiphilic polyacrylates, for example.

According to the compositions and methods of the invention, a flexible carrier may have one or more polypeptides embedded within. All that is required for a polypeptide to be considered embedded within a flexible carrier is that a portion of the polypeptide, for example, hydrophobic residues of the polypeptide, be in contact with the hydrophobic moieties such that the polypeptide is stably associated with the flexible carrier. In one embodiment of the invention, the flexible carrier may be a liposome in which a syndecan polypeptide is embedded by means of the hydrophobic interactions between the transmembrane region of the syndecan and the lipid bilayer of the liposome.

A transmembrane region is any region of a protein capable of becoming inserted or embedded into an area of hydrophobicity, for example, a lipid membrane. An area of hydrophobicity may be the lipid bilayer of a cell membrane or a liposome, for example. A transmembrane region may also be referred to as a transmembrane domain or integral membrane domain, for example. A protein comprising a transmembrane region may be referred to as a membrane protein, a transmembrane protein or an integral membrane protein, for example. Transmembrane proteins typically comprise a transmembrane domain and either an extracellular domain, an intracellular domain, or both. An extracellular domain may be referred to by other terms well known in the art, including, for example, an ectodomain. An intracellular domain of a protein expressed in a cell is in contact with the cell's cytoplasm and is therefore also called a cytoplasmic domain or a cytoplasmic tail. Transmembrane regions may comprise hydrophobic residues and/or show alpha-helical secondary structure. Methods for predicting whether a region of a protein may act as a transmembrane region are well known in the art (for example, see Cao et al., Bioinformatics, 22(3): 303-309, (2006)).

The present invention provides for the co-delivery of a molecule and a polypeptide to a cell. All that is required by the term “co-delivery” is that both the molecule and the polypeptide be delivered to a cell. Co-delivery may occur simultaneously or at discrete time points. The molecule and polypeptide may physically interact previous to the providing step or may interact subsequent to the providing step. In a preferred embodiment of the invention, the molecule is selectively bound to the polypeptide prior to the providing step.

In one embodiment of the invention, delivery of growth factors may be improved by co-delivering these growth factors with their receptors and co-receptors. Co-delivery of growth factors with receptors or co-receptors may protect growth factors from proteolysis, enhance their activity, and target the growth factors to the cell surface to facilitate growth factor signaling. In one embodiment of the invention, the syndecans may function as co-receptors in this new paradigm of drug delivery as they bind many growth factors through their heparan sulfate chains and are known to be active participants in the signaling pathways of growth factors associated with angiogenesis (e.g. Fibroblast Growth Factor (FGF) and Vascular Endothelial Cell Growth Factor (VEGF)).

In one embodiment of the present invention, syndecans may be co-delivered with growth factors. Syndecans are a class of cell surface heparan sulfate proteoglycans (HSPGs) that mediate the interaction of growth factors and their receptors. As used herein, a syndecan or fragment thereof may comprise any polypeptide containing 75% similarity to a wild-type syndecan or to a part of a wild-type syndecan that retains some biological function of a wild-type syndecan.

Generally, proteoglycans are a class of proteins that contain glycosaminoglycan (GAG) attachments. GAGs are long, unbranched polysaccharides comprising a repeating disaccharide unit. One example of a GAG is heparan sulfate. The most common disaccharide unit in heparan sulfate is glucuronic acid (GlcA) linked to N-acetylglucosamine (GlcNAc). Another example of a GAG is chondroitin sulfate, made up of the disaccharide N-acetylgalactosamine and glucuronic acid. The GAGs of proteoglycans are attached to the core proteins by a linking tetrasaccharide moiety. A glycosaminoglycan-attachment site on a syndecan protein may be any serine residue followed by a glycine residue (SG).

According to the methods of the present invention, heparanase can be supplied to a cell in addition to a molecule and polypeptide embedded in a flexible carrier. Heparanase is an enzyme that in its active form, degrades heparan sulfate chains. Heparanase is synthesized first in its inactive form, proheparanase, which consists of an 8 kDa fragment, a 50 kDa fragment and a linker region that physically links the two pro-fragments. During transport to the cell surface, heparanase is localized to the lysosome, where the linker region is excised, and the 8 kDa and 50 kDa fragments form an active dimer.

In one embodiment of the present invention, a mutant syndecan comprises a mutation in a residue recognized and/or cleaved by a sheddase, wherein the mutation decreases the ability of said mutant syndecan to be cleaved by a sheddase as compared to a corresponding wild-type syndecan. A sheddase may be any protease capable of cleaving the extracellular, or ectodomain, of a syndecan. For example, the juxtamembrane domain may be mutated to be resistant to proteolytic cleavage. For instance, one region of syndecan-1 known to be susceptible to proteolytic cleavage is the region between Gln238 and Gln252. This region could be replaced by a similar region from another syndecan that does not become cleaved, or individual cleavage sites could be mutated.

In another embodiment of the present invention, a mutant syndecan comprises a mutation in the cytoplasmic tail. For example, any serine or tyrosine can be mutated to alanine or phenylalanine to mimic a constitutive state of dephosphorylation or to aspartic acid or glutamic acid to mimic a constitutive state of phosphorylation. Other mutations can be made to affect intracelluar signaling via interactions with other proteins. For example, the C1 domain of the cytoplasmic tail of a syndecan can be mutated to affect interactions with proteins such as cortactin, src, tubulin or ezrin. The V domain can be mutated to affect interactions with proteins such as syndesmos, PKC-α, α-actinin, for example. The C2 domain can be mutated to affect interactions with proteins such as synectin, syntenin, CASK or synbindin, for example. Mutations can be made to disrupt association between a syndecan and the aforementioned proteins or other proteins known to interact with syndecans. Mutations can be made that increase association between a syndecan and the aforementioned proteins or other proteins known to interact with syndecans. Additionally, mutations can be made that alter the physical conformation of the syndecan and/or the associated protein(s) to affect the resulting process of intracellular signaling.

In addition to syndecans, many other examples of polypeptides and molecules exist that are suitable for use according to the methods and compositions of the present invention. In one embodiment, TGF receptors of type I, II, or III (including biglycan, an HSPG) and/or the co-receptors EGF-CFC, endoglin, syndecan-2 or other HSPG could be delivered together with the growth factor TGF-β to increase wound healing, particularly in patients with persistant wounds, for example, in diabetic patients. In another embodiment, the receptors PDGFRα, PDGFRβ, or any combination of the two (αα, αβ, ββ) and/or the co-receptor LRP1 may be delivered with any form of PDGF (for example, PDGF A through D) to promote wound healing. PDGF-BB (becaplermin) is currently in use as a clinical product for treating ulcers and may be used according to the methods of the present invention to alter its therapeutic efficacy. The growth factor receptor FGFR-1 and/or its co-receptors HSPGs: perlecan, syndecan 1-4, or glypican may be delivered with FGF-2 according to the methods of the present invention, to alter, for example, angiogenesis and/or wound healing. In another embodiment, the receptors VEGFR-1, VEGFR-2, VEGFR-3, neuropilin 1, or neuropilin 2 and/or the co-receptors neuropilin 1, neuropilin 2; syndecan-2 or other HSPG may be delivered with VEGF to alter, for example, angiogenesis and/or wound healing. According to the methods of the invention, plexin receptors and/or neuropilin 1 may be delivered with one or more semaphorins to alter, for example, nerve regeneration and/or neuron guidance. In another embodiment, the receptor TrkA may be delivered with NGF to alter nerve regeneration. According to the methods of the invention, the receptor EGFR and/or the co-receptors ErbB2 or ErbB3 may be delivered with the growth factor EGF to alter, for example, liver regeneration and/or wound healing. In another embodiment, the receptors LIFR (CD118) or gp30 may be delivered with Leukemia inhibitory factor (LIF) to alter, for example, nerve regeneration and/or cancer. According to the methods of the invention, bone morphogenetic protein receptors (BMPRs) and/or their co-receptors DRAGON (RGMb) or HVJ may be co-delivered with bone morphogenetic protein (BMP) to alter, for example, bone and/or cartilage regeneration. In another example according to the methods of the invention, the anti-angiogenic/cancer effect of thrombospondin may be altered by co-supplying thrombospondin, thrombospondin derived peptides or thrombospondin mimetics (such as ABT-510, currently in phase III trials) with liposome-embedded CD36 or TGFR.

Additional examples of growth factors, growth factor like peptides and cytokines include but are not limited to artemin, TGF-β family members such as transforming growth factor-β1 (TGFβ1), transforming growth factor-β (TGFβ2), transforming growth factor-β3 (TGFβ3), inhibin β A (INHβA), inhibin β B (INHβB), the nodal gene (NODAL), bone morphogenetic proteins 2 and 4 (BMP2 and BMP4), the Drosophila decapentaplegic gene (dpp), bone morphogenetic proteins 5-8 (BMP5, BMP6, BMP7 and BMP8), the Drosophila 60A gene family (60A), bone morphogenetic protein 3 (BMP3), the Vg1 gene, growth differentiation factors 1 and 3 (GDF1 and GDF3), dorsalin (drsln), inhibinα (INHα), the MIS gene (MIS), growth factor 9 (GDF-9), glial-derived neurotrophic growth factor (GDNF), neurturin (NTN), persephin, fibroblast growth factor (FGF), insulin, insulin-like growth factor I (IGF-I) or somatomedin C, insulin-like growth factor II (IGF-II) or somatomedin A, epidermal growth factor (EGF), fibroblast growth factors (acidic FGF and basic FGF), nerve growth factor (NGF), brain-derived neurotrophic factor (BDNF), neurotrophin 3 (NT-3), neurotrophin 4 (NT-4), ciliary neurotrophic factor (CNTF), hepatocyte growth factor (HCGF), transforming growth factor α (TGF-α), transforming growth factor β (TGF-β), macrophage colony-stimulating factor (M-CSF or CSF-1), granulocyte-macrophage colony-stimulating factor (GM-CSF or CSF-2), granulocyte colony-stimulating factor (G-CSF or CSF-3), platelet-derived endothelial cell growth factor (PD-ECGF), interleukins 1 to 13 (IL-1 to IL-13), interferons α, β and γ (IFN-α, IFN-β, and IFN-γ, tumor necrosis factor α (TNF-α) or cachectin, tumor necrosis factor β (TNF-β) or lymphotoxin, erythropoietin, EGF-like mitogens, TGF-like mitogens, TGF-like growth factors, PDGF-like growth factors, melanocyte growth factor (MGF), mammary-derived growth factor (MDGF-1), prostate growth factors, cartilage-derived growth factor (CDGF), chondrocyte growth factor (CGF), bone-derived growth factor (BDGF), osteosarcoma-derived growth factor (ODGF), glial growth-promoting factor (GGPF), colostrum basic growth factor (CBGF), endothelial cell growth factor (ECGF), tumor angiogenesis factor (TAF), hematopoetic stem cell growth factor (SCGF), B-cell stimulating factor 2 (BSF-2), B-cell differentiation factor (BCDF), leukemia-derived growth factor (LDGF), myelomonocytic growth factor (MDGF), macrophage-derived growth factor (MDGF), macrophage activating factor (MAF), erythroid-potentiating activity (EPA), transferrin, bombesin and bombesin-like peptides, angiotensin II, endothelin, atrial natriuretic factor (ANF), ANF-like peptides, vasoactive intestinal peptide (VIP) and bradykinin. These growth factors, growth factor-like peptides and cytokines, according to the methods of the invention, can be delivered together with their receptors and/or co-receptors to alter their therapeutic efficacy.

In another example, immunomodulatory cytokines such as IL-2, INF-α, GM-CSF, TNF-α, or IL-10 may be delivered together with their receptors or co-receptors to alter, for example, immunosuppression in an autoimmune disease or immunostimulation as an anti-cancer or anti-infection therapy.

According to the methods and compositions of the present invention, modulated cell signaling, cell proliferation, cell migration and/or cell differentiation results in modulation, control or regulation of cell, organ, or tissue preservation, repair, replacement, or regeneration, including processes that involve hypoxia, angiogenesis, wound healing, ischemia, apoptosis, or inflammation, including those of acute, reactive, autoimmune and chronic nature wound, cell, organ and tissue repair. Applicable systems include but are not isolated to repair of cosmetic or surgical wounds from superficial skin incisions, deep tissue excision or biopsies of cells, tissue or organs of the skin, hair, bones and joints (including the arthritites, degenerative, metabolic and infectious diseases), brain, eye (that might also include corneal graft rejection, neovascular glaucoma, retrolental fibroplasia, epidemic keratoconjunctivitis), ear, nose, tracheobronchial tree, oropharynx, teeth, gastrointestinal tract, salivary glands, liver, spleen, pancreas, gall bladder, genitourinary tract, kidney, bladder, uterus, ovaries, prostate accidental or unintended injury, fracture, laceration or noxious exposure diseases of the neural systems that involve tissue preservation, repair, replacement or regeneration including amyotrophic lateral sclerosis, Alzheimer's disease, Parkinson's disease Huntington's disease, ischemic stroke, acute brain injury, acute spinal chord injury, multiple sclerosis and peripheral nerve injury regeneration and guidance vascular repair and control of aneurysms, hemangiomas, thrombosis, spasm, intimal hyperplasia and restenosis, myocardial hypertrophy and remodeling, weight loss/fat metabolism, congenital dysplasia, malformation, altered development of cells, tissues and/or organs and their preservation, repair, replacement or regeneration acquired infectious diseases including bacterial, viral, parasitic and protozoal origin, and of AIDS/HIV, hematologic, neoplastic, metastatic and dysplastic diseases including cancer of solid organs, circulating blood, bone marrow and blood precursor cells and when used alone or in concert with other device, pharmacolologic, cell-based or tissue engineered therapies, including combination products and stem cell based therapies.

### Materials and Methods

### Endothelial Cell Culture

Human umbilical cord endothelial cells (HUVECs, Promocell, Germany) were cultured in MCDB 131 media (Invitrogen, Carlsbad, Calif.) supplemented with EGM-2 SingleQuot growth supplements (Cambrex Bio Science), 400 mM L-Glutamine (Invitrogen), and 5% fetal bovine serum (FBS). The cells were cultured on 100-mm culture dishes incubated at 37° C. in a humidified atmosphere of 5% CO2. HeLa cells were obtained from ATCC (Manassas, Va.) and grown in 10% FBS in DMEM at 37° C. in a humidified atmosphere of 10% CO2.

### Production of Recombinant Syndecan Protein

A constitutive expression vector containing the syndecan-4 gene (Origene, Rockville, Md.) was transiently transfected into HeLa cells using the FuGENE HD transfection reagent (Roche) per the manufacturer's specifications. After two days post-transfection, cell lysis was performed with a buffer containing the following: 20 mM Tris (pH=8.0), 150 mM NaCl, 1% Triton X-100, 0.1% SDS, 2 mM sodium orthovanadate, 2 mM PMSF, and 50 mM NaF, and protease inhibitors (Roche). The lysates were clarified by centrifugation for 15 min at 15,000 g and the supernatant was collected. The pooled lysates were desalted and separated using ion exchange and size exclusion chromatography. The samples were then desalted using dialysis. The final samples were analyzed for purity by SDS-PAGE and silver staining

### Preparation of Proteoliposomes

Stock solutions of 10 mg/ml each of 1,2-Dioleoyl-sn-Glycero-3-Phosphocholine (DOPC), 1,2-Dioleoyl-sn-Glycero-3-Phosphoethanolamine (DOPE), cholesterol and sphingomyelin (Avanti Polar Lipids, Alabaster, Ala.) were dissolved in chloroform and mixed in a ratio of 40:20:20:20, respectively. The solution was placed in a round bottom flask and the solvent removed under a stream of argon gas. The lipids were re-suspended by mixing, sonication and freeze-thawing in a HEPES buffered salt solution (10.0 mM HEPES and 150 mM NaCl in PBS, pH 7.4) to form a final solution of 13.2 mM total lipids. The lipid solution was then extruded through a 400 nm polycarbonate membrane (Avestin, Canada). A detergent, 1% n-octyl-β-D-glucopyranoside (OG), was added to both the 13.2 mM lipid and 71 μg/ml syndecan-4 protein solutions and these were combined in various ratios to form different formulations. Each of the proteoliposome solutions was incubated for one hour at room temperature with mixing. The concentration of the solution was reduced to 40% of the original in 10% increments every 30 min through dilution with PBS. The detergent and free protein was removed by extensive dialysis in PBS at 4° C. Any remaining OG was removed by repeated BioBead treatments (SM-2, Bio-Rad, USA).

### Measurement of DNA Synthesis

To study cell proliferation, a 3H-thymidine incorporation assay was done on HUVEC cells passaged and seeded onto 48-well plates. After 24 hours, the media was replaced with starvation media with 0.5% FBS. After another 48 hours, samples of the proteoliposomes were added while other groups were left with either starvation media or given FGF-2 alone. After 24 hours of treatment, one μCi/ml [methyl-³H] thymidine (Perkin-Elmer, Waltham, Mass.) was added to each well and incubated for 24 hours at room temperature. Cells were then washed 3 times with PBS at 4° C., and precipitated with 10% trichloroacetic acid at 4° C. for 30 min. After being washed twice with 95% ethanol, the cells were solubilized with 1 ml of 0.25M NaOH with 0.1% SDS and then neutralized with 1M acetic acid. Beta emission was measured using liquid scintillation.

### Wound Edge Migration Assay

Plates of confluent HUVEC cells were wounded with the edge of a cell scraper, and the boundaries of the wound marked on the underside of the plates using a hypodermic needle. The dishes were washed three times with serum-free media and solutions of FGF-2 (10 ng/ml) with various liposome formulations were applied. The wounds were photographed using an inverted, phase contrast microscope with digital camera (Nikon D50) and migration distance quantified using Photoshop CS3 (Adobe, San Jose, Calif.).

### Angiogenesis Assay

In-vitro tube formation was measured using an In-Vitro Angiogenesis Assay Kit (Millipore, Billerica, Mass.). Briefly, 6-well culture plates were coated with matrigel and allowed to gel overnight at 37° C. To each well 2×104 endothelial cells were added in the presence of the appropriate treatment (i.e. liposome/FGF formulation). At various time points the cells were imaged using phase contrast microscopy. Quantification of tube length and branch points was performed using MetaMorph software (Molecular Devices, Sunnyvale, Calif.).

### Rat Hind Limb Ischemia Model

The rat hind limb ischemia model was performed as previously described. Sprague Dawley rats were anesthetized using isofluorane gas. A 1-cm longitudinal incision was made over the inguinal region of the right hind limb. The femoral artery was separated from the femoral nerve and vein and ligated twice using surgical silk. Using blunt dissection, a pocket was created in the subcutaneous space and a small osmotic pump (DURECT Corporation, Cupertino, Calif.) was implanted containing 5 μg of FGF-2 with various co-delivery formulations. This pump was designed to deliver the entire volume of 100 μl over a period of 14 days. The incision was then sealed using surgical clips. After 7 days the animals were sacrificed, the hind limb muscles were harvested and then frozen in liquid nitrogen. All animal experiments were performed in accordance with the Guide for the Care and Use of Laboratory Animals published by the U.S. National Institutes of Health (NIH Publication No. 85-23).

### Histological and Immunohistochemical Analysis

Harvested muscle and skin samples were sectioned at 8 μm using a cryotome equipped with a steel knife. Prior to hematoxylin and eosin (H&E) staining, the sections were dried and fixed in formalin for 10 min. Histological staining was then performed using standard methods. For immunohistochemical staining for PECAM, the sections were air dried and fixed in acetone at −20° C. for 2 minutes. The samples were then blocked with 20% FBS for 45 minutes and exposed to a 1:25 dilution of goat anti-PECAM 1 (Santa Cruz Biotechnology, Santa Cruz, Calif.) for 2 hrs. The samples were washed three times with PBS and treated with a 1:100 dilution of secondary antibody conjugated to a fluorescent marker (Alexa Fluor 594, Invitrogen) for one hour. The sections were then rinsed with PBS and coverslipped with DAPI containing anti-fade mounting medium (Vector Labs, Burlingame, Calif.).

### EXAMPLES

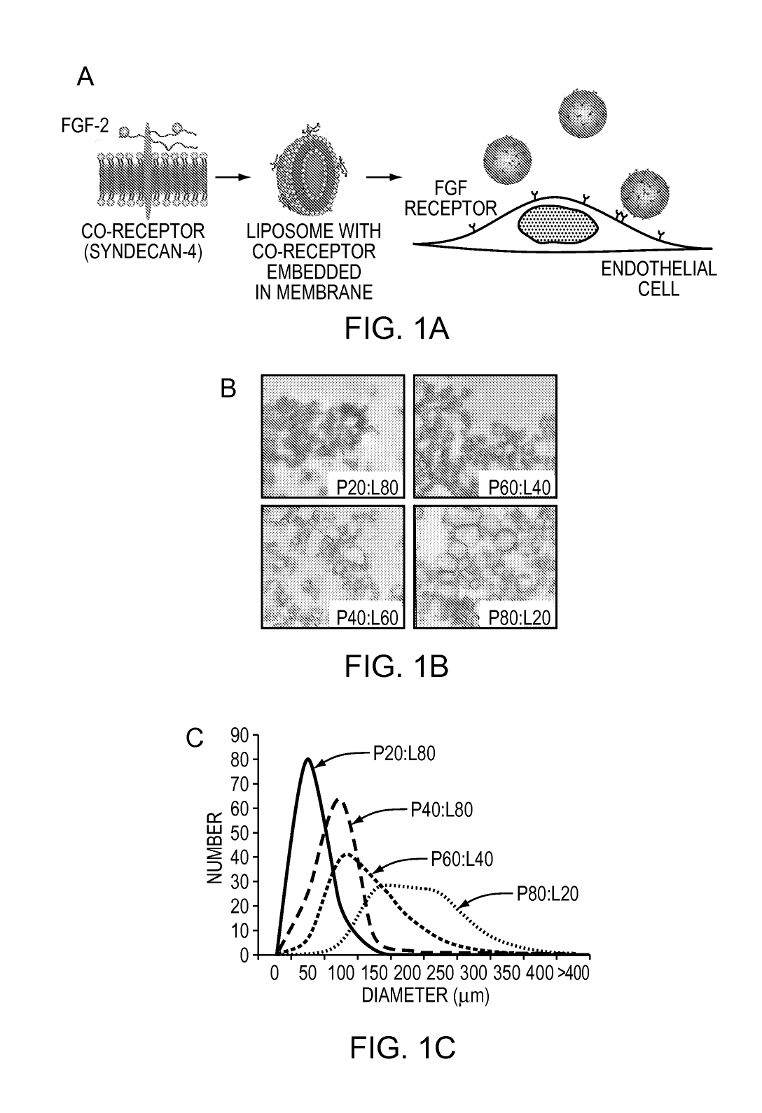

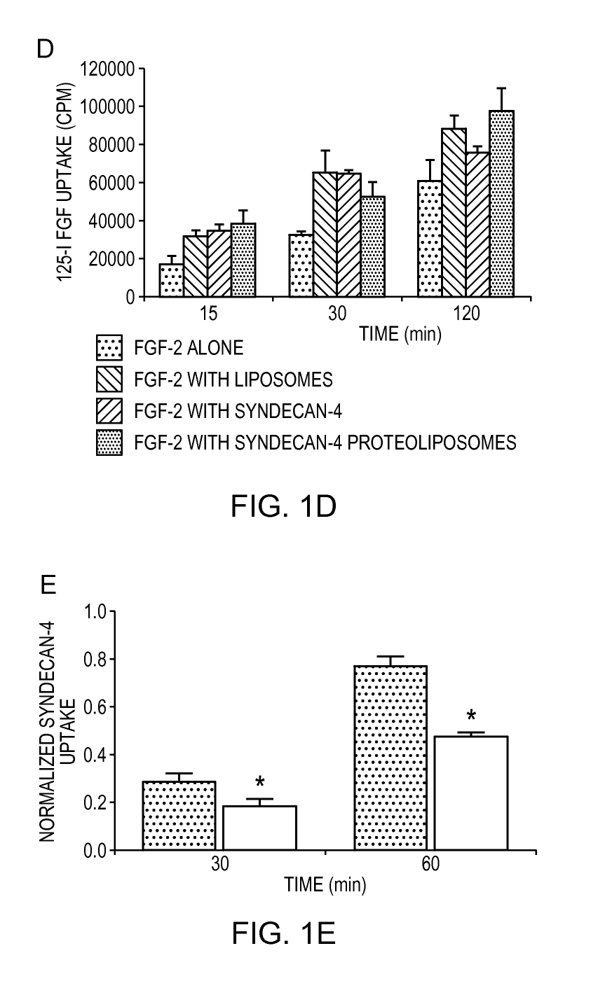

Recombinant syndecan-4 were produced by transfecting HeLa cells with a constitutive expression vector for syndecan-4 and purifying with chromatography. A detergent was added to this purified protein and to unilamellar liposomes produced through extrusion. The protein and liposomes were combined and the detergent was removed through slow, progressive dilution, dialysis and zeolite-based absorption. Transmission election microscopy revealed that the final size distribution of the liposome was dependant upon the syndecan-4 to lipid ratio with larger liposomes forming from the solutions with higher protein content (FIGS. 1b and 1c). We performed an analysis of uptake of ₁₂₅I-FGF-2 in endothelial cells and found that both the presence of liposomes or syndecan-4 alone enhanced FGF-2 uptake (FIG. 1d). Liposome embedded syndecan-4 caused the greatest increase in FGF uptake leading to a 2.3 fold and 1.6 fold enhancement of uptake after 15 minutes and 120 minutes, respectively (p<0.05). We metabolically labeled syndecan-4 using a mixture of tritiated amino acids and found that liposome embedding stabilized the presence of syndecan-4 within the soluble milieu (FIG. 1e).

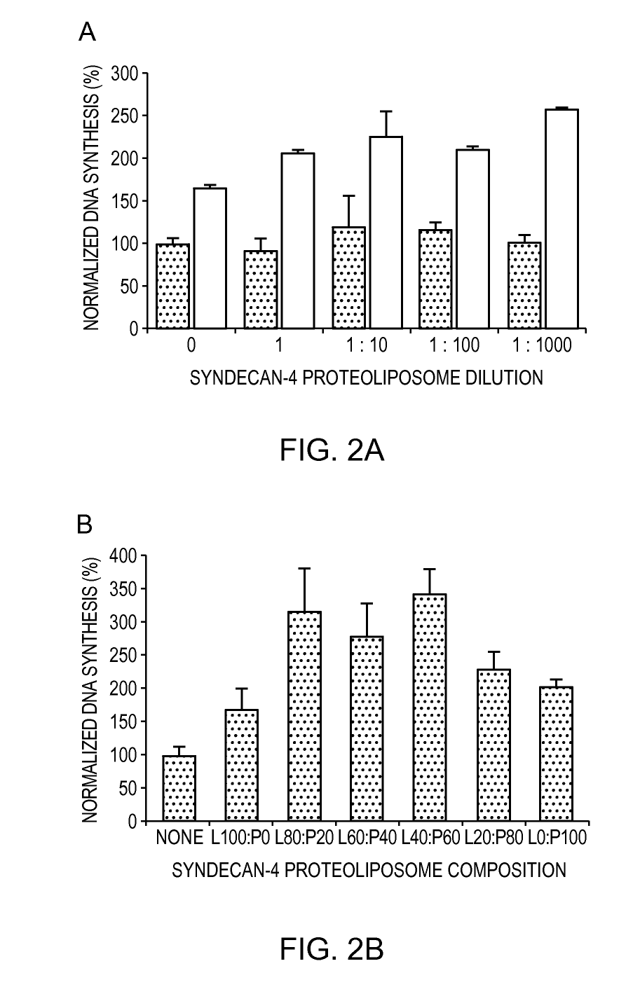

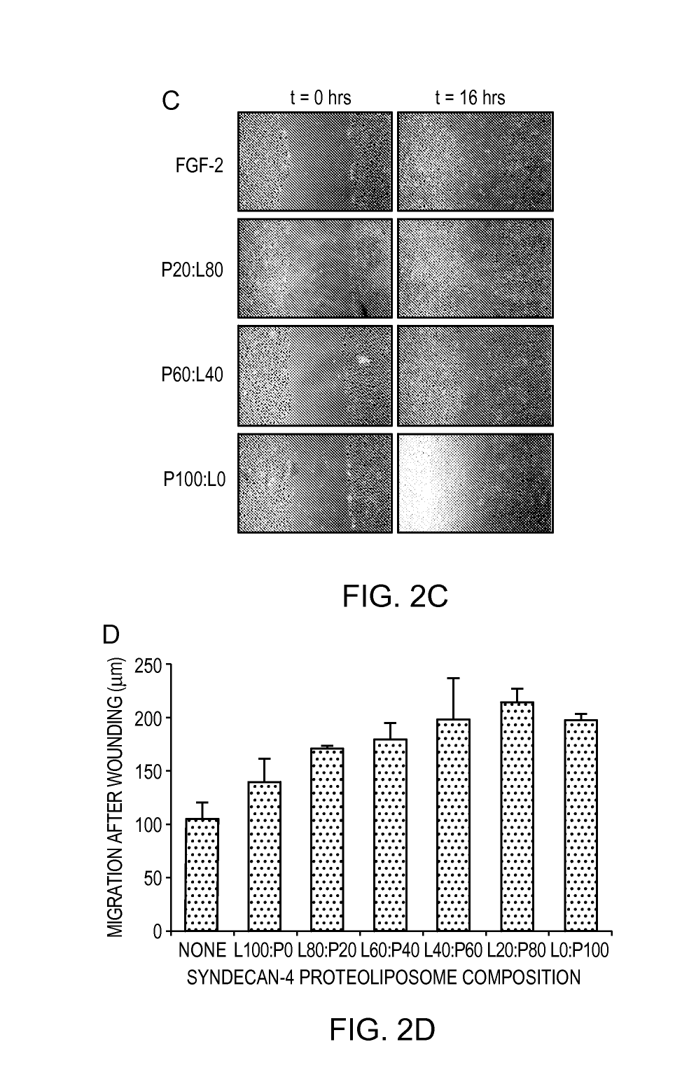

Fibroblast growth factor-2 (FGF-2) is a mediator of proliferation in endothelial cells. We performed a dose response to examine the effect of liposome-incorporated syndecan-4 concentration on endothelial DNA synthesis in the presence of constant levels of FGF-2. The addition of the liposome/syndecan-4 construct led to over a twofold enhancement in the proliferation of endothelial cells which remained constant over a wide range of treatments (FIG. 2a). Liposome embedded syndecan-4 did not enhance proliferation in the absence FGF-2 and had no toxicity at relatively high concentrations. We examined the ratio of protein to lipid in altering the effectiveness of enhancing FGF activity and found a broad range of increased activity from liposomes with protein to lipid ratios from 80:20 to 20:80 (lipid:protein; FIG. 2b). Overall, the liposome/syndecan-4 delivery system when optimized for both dose and composition led to an increase in ³H-thymidine incorporation of approximately 3.4 times that of FGF-2 alone. The mitogenic properties of our system were examined by wounding monolayers of confluent endothelial cells in the presence of FGF or FGF in combination with liposome/syndecan-4 of varying composition (protein to lipid ratio). This analysis demonstrated enhancement of migration in endothelial cells with liposomes having higher concentrations of protein and with syndecan-4 protein alone (FIGS. 2c and 2d). Stimulation of in-vitro wound healing led to an increase in wound edge migration rate about twice as fast as FGF-2 alone.

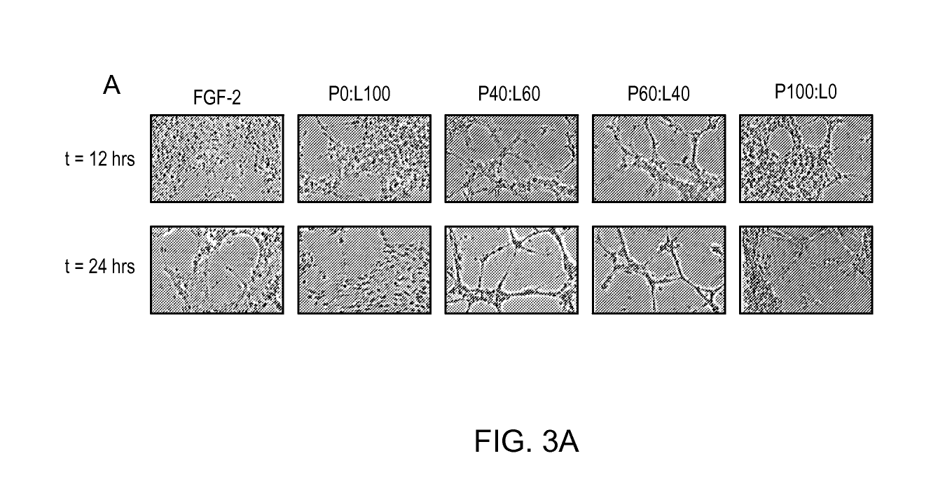

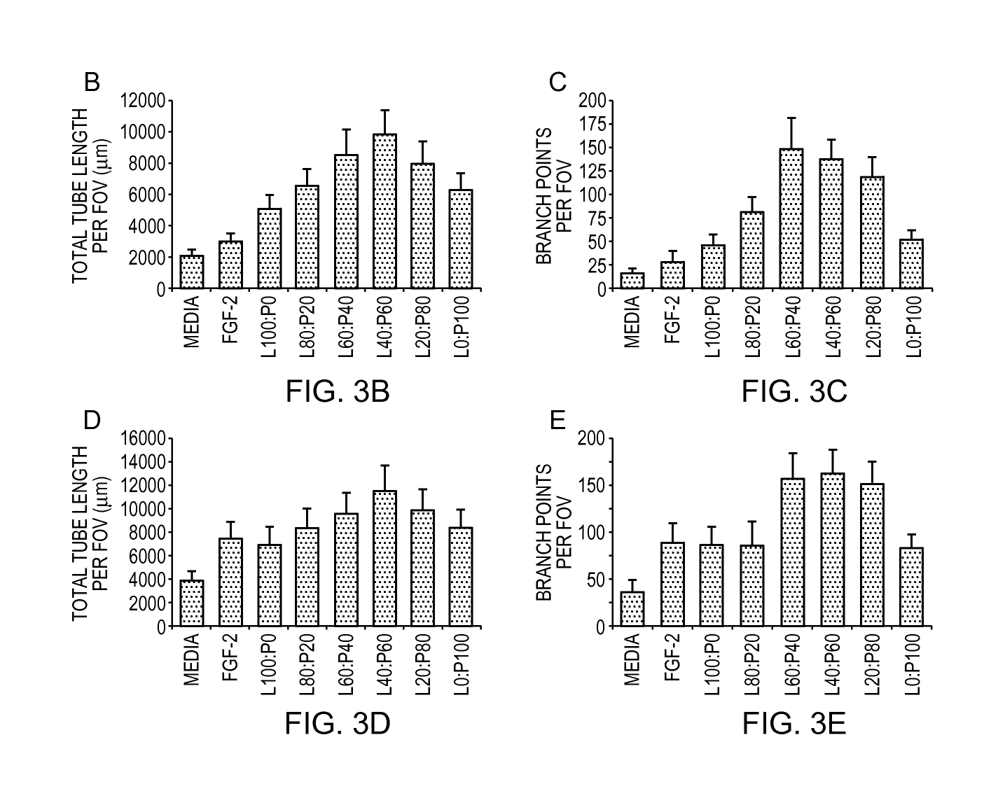

After demonstrating in-vitro activity for our system in enhancing FGF induced proliferation and mitogenesis, we examined the effects of liposome embedded syndecan-4 in enhancing FGF mediated angiogenic differentiation in an in-vitro tube formation assay. Endothelial cells were seeded in culture plates coated with Matrigel and then exposed to FGF with various liposome/syndecan-4 formulations (FIG. 3a). Mid-range composition liposome/syndecan-4 constructs were effective in enhancing FGF-2 activity in stimulating angiogenesis, leading to a 9.9-fold increase in tube length and 4.7-fold increase in branch points after 12 hours of FGF exposure (FIGS. 3b & 3c). This enhancement was maintained after 24 hours, with a 2.9-fold increase in tube length and 4.4-fold increase in branch points (FIGS. 3d and 3e).

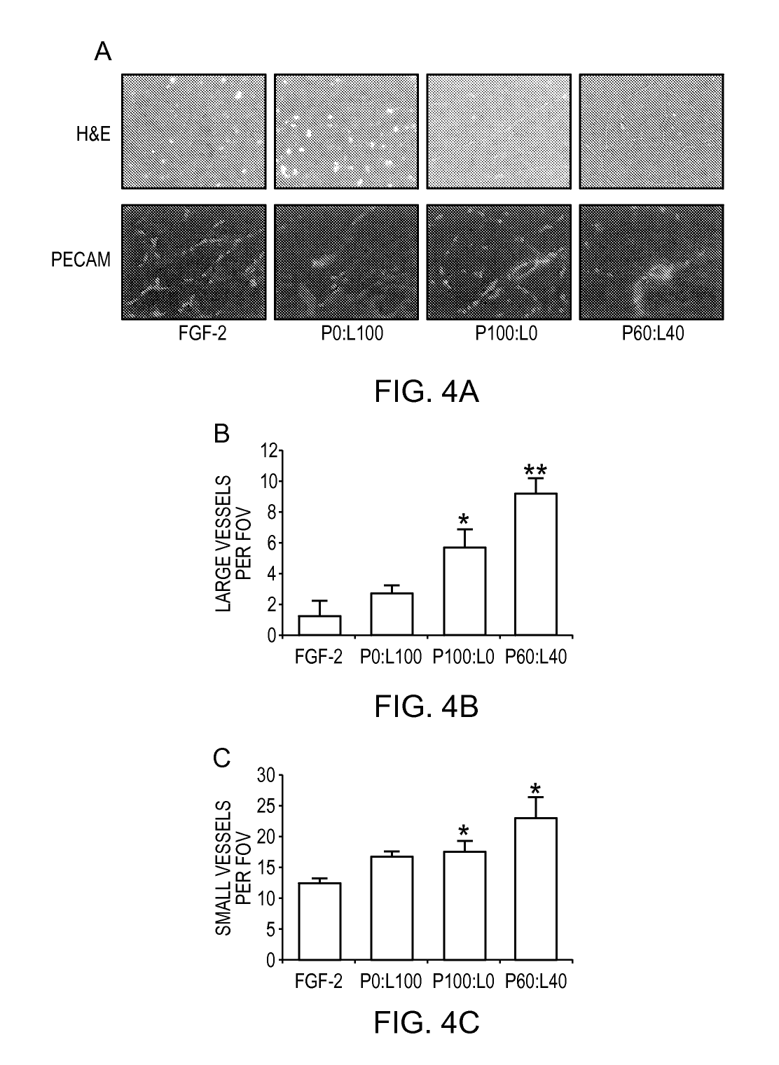

Exogenously delivered FGF-2 has been shown to enhance revascularization of limbs following ischemia. We tested whether our formulation could lead to enhanced revascularization using the hind limb ischemia model in rats over the level induced by FGF-2 alone. We created ischemia in the rat hind limb by ligating the femoral artery. An osmotic pump was used to deliver FGF-2 or FGF-2 in combination with lipid alone, protein alone or the combination of the two. Following seven days of ischemia the liposome embedded syndecan-4 and syndecan-4 alone treatment groups qualitatively less ischemic change to the hind limb muscle (FIG. 4a). Staining for the endothelial marker PECAM and morphometric analysis showed a 7.3 fold increase in arterioles (FIG. 4b) as well as a 1.9 fold increase in capillary density (FIG. 4c). These data show a marked improvement not over untreated ischemia but over FGF-2 treatment alone.

In the clinical domain, ischemia most commonly results from the effects of microvessel and macrovessel atherosclerotic disease. Existing co-morbidities such as diabetes, old age and hypertension are present in a large portion of these patients and compromise the revascularization potential of peripheral and myocardial tissues. While prior work has focused on delivering recombinant proteins, genes or cells that can facilitate angiogenesis, little has been done on examining ways in which to improve cell response to delivered growth factors. In this work we have demonstrated a novel method for increasing the in-vitro and in-vivo activity of growth factors. Our concept was to deliver, in addition to a growth factor, a co-receptor embedded in liposomes which would prime the cell to respond more robustly to growth factor stimulation and prevent some forms of growth factor desensitization. To this end, we have demonstrated that liposome embedded syndecan-4 can facilitate cellular uptake of FGF-2 and increase endothelial cell proliferation, migration and angiogenic differentiation. Further, when applied to a rat model of hind limb ischemia, liposome embedded syndecan-4 caused increased angiogenesis and arteriogenesis in comparison to FGF-2 alone.

In order for growth factor therapy to be performed by the method of direct protein delivery several criteria must be met. The drug must be delivered to the ischemic region in an appropriate concentration and without degradation by proteases. Because cells in general respond more strongly to prolonged growth factor exposure, controlled release or multiple injection strategies must be used to obtain revascularization. The hydrophilic nature of the growth factors and the immense binding capacity of local HSPGs can limit diffusion of growth factor through tissue, potentially reducing the therapeutic region. The ischemic myocardium and peripheral tissue are known to have enhanced protease activity, potentially leading to growth factor degradation. The syndecans themselves are highly vulnerable to protease-induced shedding from the cell surface. Secondly, a growth factor must be able to effectively induce signaling in the cell. This requires the cell to have an exposed cell surface receptor and, in the case of FGF-2, the presence of a heparin or heparan sulfate to stabilize the signaling complex. Following binding and complex formation the cell must retain the ability to do intracellular signaling and be able to respond appropriately. The presence of HSPGs and heparanase is altered by diabetes, hyperlipidemia, ischemia and surgical interventions. Our delivery formulation has many advantages with respect to these steps. Associating the FGF-2 with liposomes increases the overall hydrophobicity and consequently diffusion based penetration through tissue. In our studies, liposomes alone appeared to also aid uptake of FGF-2 into the cell and preserve syndecan-4 in the culture medium. The presence of liposomes may facilitate FGF-2 entry into the cell by altering lipid raft formation dynamics or by directly allowing FGF entry during liposome uptake. Another advantage of this system is the ability to add receptors or co-receptors that are not present in the target cell to enhance signaling. In this case, syndecan-4 is present in endothelial cells but the syndecan-4 we delivered was produced in a cancer cell line. As a result of their cancerous properties, these cells add heparan sulfate chains that are extremely efficient at enhancing FGF-2 signaling and migration. This method allows us to take advantage of the pro-growth nature of the cancer cells to create a highly efficient FGF-2 signaling on the endothelial cells.

Prior to this work people have only used growth factors or transfected growth factor receptors into cells or tissues to try to enhance neovascularization. One aspect of this work is that the liposomal incorporation of the co-receptor is superior to the receptor itself. This effect was present in our studies on proliferation, tube formation and revascularization in hind limb ischemia. This speaks to the advantages of incorporating hydrophobicity into a drug delivery system, especially in the case of receptors in which membrane and surface association is fundamental. Hydrophobic drugs have enhanced tissue deposition and have reduced washout. We also found that efficacy of liposome embedded syndecan-4 is relatively insensitive to the concentration of the delivered compound. One might expect syndecan-4 to serve, at high concentrations, to competitively compete with receptor binding. This indeed may be the case with the soluble receptor alone but in our studies the addition of the liposomal carrier appeared to abolish this effect. The relative insensitivity to dosing is an advantage for delivery, providing a rigorous biological response even in the face of altering levels of drug that have diffused from a local delivery or injection site. An intriguing finding, as well, is the enhancement of FGF-2 migration in endothelial cells. We can therefore see that addition of syndecan-4, independent of incorporation into proteoliposome, can enhance migration in the presence of FGF. These results contrast with results found for the proliferation and in-vitro tube formation studies in which having a nearly equal ratio between lipid and protein was effective. In addition to being a co-receptor for FGF-2, syndecan-4 also has a role in cell attachment. Syndecan-4 is an essential component for the activation of focal adhesion kinase and can bind fibronectin with its heparan sulfate chains. For these studies, we are likely observing the combined effects of delivering both an enhancer of FGF-2 signaling and an exogenously delivered adhesion receptor.

Here we have presented evidence that delivery of co-receptor proteoliposomes can enhance the cellular efficacy of a delivered agent. This conceptual paradigm of delivering a receptor or co-receptor to increase cellular response may be applicable to a wide variety of therapeutic applications that are amenable growth factor and cytokine therapy. We used the syndecan-4/FGF-2 system to demonstrate this archetype of therapy and have shown enhancement of in-vitro proliferation, migration and differentiation of endothelial cells as well as the in-vivo enhancement of neovascularization in the ischemic hind limb. The results presented here represent the first report of recombinant receptors being delivered to enhance angiogenesis. In the context of treatments for clinical ischemia, it is clear that delivery of recombinant FGF or VEGF as it is currently performed is not sufficient to achieve efficacy in diseased patients and this strategy may facilitate the development of more effective forms of therapeutic neovascularization.

1. A method for modulating the therapeutic efficacy of a molecule, said method comprising the steps of:
(a) providing a flexible carrier comprising a hydrophobic moiety and with at least one polypeptide embedded therein, said at least one polypeptide comprising a transmembrane region that is in contact with the hydrophobic moiety of the flexible carrier and is stably associated with the flexible carrier; and
(b) co-delivering to a cell (i) a molecule capable of selectively binding said at least one polypeptide and (ii) the flexible carrier into which said at least one polypeptide is embedded, wherein the flexible carrier facilitates co-delivery to the cell membrane, and wherein said co-delivery results in modulation of the therapeutic efficacy of said molecule.

2. A method for modulating cell signaling, cell secretion, cell proliferation, cell migration and/or cell differentiation, said method comprising:
(a) providing a flexible carrier comprising a hydrophobic moiety and with at least one polypeptide embedded therein, said at least one polypeptide comprising a transmembrane region that is in contact with the hydrophobic moiety of the flexible carrier and is stably associated with the flexible carrier; and
(b) co-delivering to a cell (i) a molecule capable of selectively binding the at least one polypeptide and (ii) the flexible carrier into which the at least one polypeptide is embedded, wherein the flexible carrier facilitates co-delivery to the cell membrane, and wherein said co-delivery results in modulation of cell signaling, cell proliferation, cell migration and/or cell differentiation.

3. The method of claim 2, wherein said modulated cell signaling, cell proliferation, cell migration and/or cell differentiation results in modulation, control or regulation of cell, organ, or tissue preservation, repair, replacement, or regeneration.

4. The method of claim 1 or 2, wherein co-delivery of said molecule and of said flexible carrier into which the at least one polypeptide is embedded occurs simultaneously.

5. The method of claim 1 or 2, wherein the at least one polypeptide comprises a syndecan or fragment thereof.

6-8. (canceled)

9. The method of claim 1 or 2, wherein the at least one polypeptide comprises wild-type syndecan-4 or a fragment thereof.

10-21. (canceled)

22. The method of claim 1 or 2, wherein the molecule is a growth factor.

23-24. (canceled)

25. The method of claim 1 or 2, wherein the flexible carrier comprises two polypeptides, wherein said two polypeptides are a growth factor receptor and a syndecan, and further wherein said molecule is a growth factor.

26. The method of claim 1 or 2, wherein the flexible carrier comprises lipids and proteins.

27. The method of claim 26, wherein the ratio of said lipids to said proteins is in the range from 20:80 to 80:20.

28. The method of claim 26, wherein the flexible carrier comprising lipids and proteins is a liposome.

29. A method for modulating cell signaling, cell secretion, cell proliferation, cell migration and/or cell differentiation, said method comprising:
(a) providing a liposome comprising a hydrophobic moiety and with syndecan-4 embedded therein, said syndecan-4 comprising a transmembrane region that is in contact with the hydrophobic moiety of the liposome and is stably associated with the liposome; and
(b) co-delivering to a cell (i) fibroblast growth factor (FGF) and (ii) the liposome comprising syndecan-4, wherein the liposome facilitates co-delivery to the cell membrane, and wherein said co-delivery results in modulated signaling, secretion, proliferation, migration and/or differentiation of said cell.

30. The method of claim 29, wherein said modulated cell signaling, cell proliferation, cell migration and/or cell differentiation results in modulation, control or regulation of cell, organ, or tissue preservation, repair, replacement, or regeneration.

31-32. (canceled)

33. A method for enhancing angiogenesis, said method comprising the steps of:
(a) providing to a subject a flexible carrier comprising a hydrophobic moiety and with a syndecan and/or a growth factor receptor embedded therein, said syndecan-4 and/or a growth factor receptor comprising a transmembrane region that is in contact with the hydrophobic moiety of the flexible carrier and is stably associated with the flexible carrier; and
(b) co-delivering to said subject (i) a growth factor capable of selectively binding said syndecan and/or said growth factor receptor and (ii) the flexible carrier into which said syndecan and/or said growth factor receptor is/are embedded, wherein the flexible carrier facilitates co-delivery to said subject's cell membrane, wherein said co-delivery results in enhancement of angiogenesis.

34. The method of claim 33, wherein said subject has peripheral or myocardial ischemia.

35-44. (canceled)

45. The method of claim 22, wherein the growth factor is fibroblast growth factor 2 (FGF-2).

46. The method of claim 2 or 29, wherein said co-delivery results in angiogenesis in a subject that has ischemia.

47. (canceled)
